(19) United States
(12) Patent Application Publication
(10) Pub. No.: US 2011/0224646 A1
(43) Pub. Date: Sep. 15, 2011

(54) Methods and Devices for Tailoring a Bolus Delivery Pattern

(76) Inventors: Ofer Yodfat, Modi'in (IL); Gali Shapira, Haifa (IL); Aharon Levy, Moshav Beit Hanan (IL)

(21) Appl. No.: 13/063,677

(22) PCT Filed: Sep. 10, 2009
(86) PCT No.: PCT/IL2009/000888

§ 371 Date: May 24, 2011

Related U.S. Application Data
(60) Provisional application No. 61/096,087, filed on Sep. 11, 2008.

(51) Int. Cl. A61M 5/168

(52) U.S. Cl. 604/504; 604/66

(57) ABSTRACT

Methods and devices for delivering therapeutic fluid to the body are described. The method may include determining a bolus delivery pattern based on a specific dietary intake of a patient and delivering the therapeutic fluid into the patient's body based, at least in part, on the determined bolus delivery pattern. The devices used in conjunction with the disclosed methods may include a controller to determine a bolus delivery pattern based on a specific dietary intake of the patient and an infusion pump to deliver the therapeutic fluid into the patient's body based, at least in part, on the determined bolus delivery pattern.

### CROSS-REFERENCE TO RELATED APPLICATIONS

The present disclosure claims priority to U.S. Provisional Patent Application No. 61/096,087, entitled “Method and a Device for Tailoring a Bolus Delivery Pattern in a Drug Delivery Device,” filed on Sep. 11, 2008, the disclosure of which is incorporated herein by reference in its entirety.

### FIELD

Methods, devices and systems for sustained delivery of therapeutic fluids (e.g., insulin) to a user/patient are described. In particular, the present disclosure includes embodiments directed toward portable devices and methods for implementing these devices for tailoring a bolus delivery pattern of therapeutic fluid. More particularly, skin securable insulin dispensing devices and methods for implementing these devices for tailoring a bolus delivery pattern according to the nutritional characteristics (e.g., amount of carbohydrates, glycemic index or fat content) of an individuals' dietary intake are provided.

### BACKGROUND

Diabetes mellitus is a disease of major global importance, increasing in frequency at almost epidemic rates, with 170 million known cases worldwide in 2006. This number is expected to at least double over the next 10-15 years. Diabetes is characterized by a chronically raised blood glucose concentration (known as hyperglycemia) due to a relative or absolute lack of the pancreatic hormone, insulin. In a healthy (i.e., non-diabetic) pancreas, beta cells located in the islets of Langerhans continuously produce and secrete insulin according to blood glucose levels and maintain near constant glucose levels in the body.

Much of the burden of the disease to the patient and health care resources is due to the long-term tissue complications that affect both small blood vessels (i.e., microangiopathy causing eye, kidney and nerve damage) and large blood vessels (causing accelerated atherosclerosis with increased rates of coronary heart disease, peripheral vascular disease and stroke). The Diabetes Control and Complications Trial (“DCCT”) has demonstrated that development and progression of the chronic complications of diabetes are greatly related to the degree of altered glycemia as quantified by determinations of glycohemoglobin (HbA1c). See, e.g., DCCT Trial, New Eng. J. Med. 1993, 329: 977-986; UKPDS Trial, Lancet 1998, 352: 837-853; BMJ 1998, 317, (7160): 703-13; EDIC Trial, New Eng. J. Med. 2005, 353, (25): 2643-53. Thus, maintaining euglycemia by frequent glucose measurements and making corresponding adjustments of insulin delivery is of utmost importance.

Insulin pumps can deliver rapid acting insulin 24 hours a day through a catheter placed under the skin. The total daily insulin dose includes basal and bolus doses. Insulin basal doses may be delivered continuously (or periodically) over a 24-hour period and facilitate keeping the blood glucose levels in an acceptable insulin range between meals and overnight. Diurnal basal rates can be pre-programmed or manually changed according to various daily activities.

Insulin bolus doses may be delivered before or after meals to counteract carbohydrate loads and/or during episodes of high blood sugar levels. The amount of insulin in a bolus dose may depend on several parameters:
the amount of carbohydrates in the meal to be consumed;
the carbohydrate-to-insulin ratio (“CIR”), i.e. the amount of carbohydrates balanced by one unit of insulin;
insulin sensitivity (“IS”), i.e. the blood glucose value lowered by one unit of insulin;
the current blood glucose level (“CBG”);
the target blood glucose level (“TBG”), i.e. the desired blood glucose level. TBG for most people suffering from diabetes is in the range of 80-130 mg/dL; and
the remaining insulin (“RI”, also referred-to as “residual insulin”), i.e. the amount of stored insulin remaining active in the body after earlier bolus doses have been delivered. This parameter is relevant when there is a short time interval between delivering consecutive bolus doses (e.g., intervals of less than 5 hours).

The insulin amount in a delivered bolus dose can be determined using equations that incorporate the above parameters, as described, for example, in U.S. Pat. No. 6,936,029 to Mann et al., the disclosure of which is incorporated herein by reference in its entirety. The insulin amount may also be determined using a method as described in co-owned, co-pending U.S. publication no. 2008/0234663, the disclosure of which is incorporated herein by reference in its entirety. It should be noted that the above methods for determining the bolus dose to be delivered generally involve assessing a bolus dose but do not address the manner and/or pattern for delivering the dose.

A bolus dose delivery pattern may refer to a rate or rates at which a bolus dose is delivered over time. For example, a meal containing purely carbohydrates may require very fast delivery at one constant delivery rate. A meal containing carbohydrates and fat (e.g., a pizza meal) may require a fast delivery rate for a short time followed by a slower delivery rate for an extended time period (e.g., two delivery rates).

Certain parameters, such as the glycemic index (“GI”) or fat content of the consumed food, can influence carbohydrate absorption and consequently bolus delivery duration and pattern. The GI can be described in terms of a ranking system based on the type of carbohydrates contained in food and their effect on blood glucose levels. According to the GI, glucose (the fastest-acting carbohydrate) is given a value of 100, and the other carbohydrates are ranked relative to that value. Ripeness, cooking time, fiber, and fat content in the food can all impact the GI of certain foods. As shown in FIG. 1, different foods have different GI's.

A low GI food will release glucose more slowly and steadily. A high GI food will release glucose more rapidly. A meal containing a carbohydrate load having high GI will thus require immediate insulin delivery to counteract the rapidly absorbed carbohydrates. Conversely, a meal containing a carbohydrate load having a low GI will require insulin delivery over an extended time period to counteract the slowly absorbed carbohydrates. In addition, changes in blood glucose levels following food consumption and/or delivery of an insulin dose may vary from one diabetic patient to another.

Currently, most insulin pumps enable the user to program a bolus dose delivery pattern before delivery. The most common programmable bolus dose delivery patterns include:
immediate (“normal”)—the entire bolus dose is delivered at the fastest pump delivery rate;
extended—the entire bolus dose is delivered over a period of time (e.g., 30 min, to 8 hours) at a constant rate; or
combined (“dual wave”)—some of the bolus dose is delivered immediately and the rest of the dose is delivered as an extended bolus dose. The ratio between the immediate portion and the extended portion may be selected by the patient as described in U.S. Pat. No. 6,852,104 to Blomquist, the content of which is incorporated herein by reference in its entirety.

In conventional systems, a user may program a bolus dose delivery pattern by inputting data, such as the duration of the bolus dose delivery (for an extended bolus or extended bolus portion of a combined bolus) and/or the ratio between the immediate bolus portion and extended bolus portion (for a combined bolus).

A drawback associated with users preprogramming bolus dose delivery information is the user's inability to estimate the appropriate bolus delivery duration and pattern (e.g., immediate, extended or combined). Often, a user's decision about the bolus dose delivery pattern can be arbitrary and based merely on intuition.

In addition, mechanical capabilities of the pump, which can enable minute-by-minute variability in delivery rate, is not fully being exploited, as the most “complex” bolus dose delivery pattern currently offered to users is composed of merely two phases in the combined or “dual wave” bolus dose.

### SUMMARY

Devices and methods for providing a user with a bolus delivery pattern (hereinafter “bolus delivery pattern”) tailored to the user's physiology are described. In some embodiments, the physiological parameters may correspond to at least one of a heart rate, a ventilation rate, a body temperature, insulin absorption characteristics (e.g., rate), physical activity (e.g., exercise of the user per period of time), cannula insertion site or fat tissue characteristics of the patient. In some embodiments, the devices and methods may provide a diabetes patient with better treatment and care by tailoring bolus delivery patterns to emulate a non-diabetic person's average blood glucose (“BG”) response to food consumption. Some methods and devices may provide for tailoring bolus delivery patterns of a drug. Preferred method aspects include tailoring a bolus delivery pattern of a drug by comparing a non-diabetic person's average BG response over time (hereinafter “healthy response”) to that of the user after consuming a similar meal and correcting the user's response to match the healthy response. In some method embodiments, the bolus delivery pattern may be tailored for a user by analyzing the physiological post-prandial increase in BG observed in one or more non-diabetic individuals. Such methods contrast with the objectives and functionality of pumps currently available to users, which bring users to fasting target blood glucose levels without considering any physiological post-prandial increases in BG observed in a non-diabetic individual.

Some method embodiments for providing a user with tailored bolus delivery patterns may include using graphs representing the healthy response to different meals (e.g., 4-5 meal types of different GI's). The “y” axis of the graph may represent the measured BG (mg/dL) and the “x” axis may represent the time that has elapsed since the meal (e.g., in minutes). Subsequently, the area-under-the-curve (hereinafter “AUC”) of the average healthy response (“AUCₕ”) may be calculated. The AUC may be presented in units of [min*mg/dL]. BG measurements may be performed at constant time intervals (e.g., every 20 min.) to determine BG versus time curves. When the user subsequently consumes similar meals (e.g., 4-5 meal types), an adequate bolus dose (e.g., based on adequate carbohydrate load assessment and the user's CIR), preferably an extended bolus (e.g., 60 min.) in some embodiments, can be administered to counteract the content of meals, starting at a predefined period before the meal (e.g., 30 min.). Mathematical relationships (which may be represented as graphs) representing the user's response to the different meals, after the bolus delivery may be provided. The BG may be measured periodically (e.g., every 20 min.) to obtain the mathematical relationship (e.g., a graph). The AUC of the user's response (“AUCᵤ”) can then also be computed. In some embodiments, the optimal, or near optimal, dose and dose distribution rate over a particular period of time (i.e., the delivery rate) can be determined from the difference between the AUC of the user and the AUC of a healthy response, i.e., AUCᵤ-AUCₕ.

In some embodiments, a drug delivery device may be implemented to perform a bolus delivery pattern tailoring operation to adjust a bolus delivery rate to a predefined meal or meal type. Some embodiments of the device may include a processor configured to control the bolus dose delivery pattern tailoring operation. In some embodiments, the device may also include a memory configured to store one or more bolus delivery patterns according to one or more meals or meal types. A display for providing graphical representations of the tailored bolus delivery pattern may be included on the device according to some embodiments. Some device embodiments may also have a user interface to receive a meal or meal type input from the user, where the meal or meal type input corresponding to a tailored graphical bolus delivery pattern. In some embodiments, the drug may be insulin and the drug delivery device may be an insulin pump.

In some embodiments, a device that can monitor BG levels and dispense bolus doses according to a tailored bolus delivery pattern is provided. In some embodiments, a device that continuously (or periodically) monitors BG levels and concomitantly delivers bolus doses according to a tailored bolus delivery pattern is provided.

In some embodiments, a device that includes a dispensing patch unit composed of two parts—a reusable part and disposable part—may be provided. The reusable part may contain relatively expensive components and the disposable part may contain relatively inexpensive components, thus providing a low cost product for the user and an economically viable product for the manufacturer and/or payer (e.g., healthcare insurance providers). In some embodiments, the unit may be skin securable. The device may also infuse insulin. The device may further be implemented to perform a procedure for tailoring an insulin bolus delivery pattern for a user. The dispensing patch unit may be attached to the skin directly or via a cradle unit. In some embodiments, the device may be remotely controlled. Some embodiments provide a device with a dispensing patch unit that can be disconnected and/or reconnected to a user. The device may implement a procedure for tailoring a bolus delivery pattern, according to some embodiments. Some embodiments disclosed herein may also provide a device having a miniature skin securable dispensing patch unit that can continuously dispense insulin and monitor BG levels. This device may also implement a procedure for tailoring a bolus delivery pattern.

Some embodiments disclosed herein may provide a semi-closed loop system that can monitor BG levels and dispense bolus doses according to sensed glucose levels and a tailored bolus delivery pattern. The system may be implemented in a single miniature device that may be discreet and cost-effective for users and/or payers (e.g., health insurance companies).

In some embodiments, the method(s) for providing a suitable bolus delivery pattern can be based on re-establishing tailored bolus profiles, for example, by comparing a user's BG responses to certain meals to a profiled healthy response to the same meals and providing an appropriate pre-established tailored bolus delivery pattern in subsequent meals of the same type. The data that determines the meal type may include at least the GI of the food to be consumed. In some embodiments, the tailored bolus delivery pattern can be selected from a plurality of pre-established bolus delivery patterns, with each pattern allocated to a different GI range (e.g., GI<55, 56<GI<69, GI>70). The GI of the food may be designated as a qualitative, descriptive parameter (“QDP”) (e.g., high GI, intermediate GI, low GI or combined GI). Presenting the GI of the food as a qualitative parameter instead of a number may be particularly convenient for young children.

In some embodiments, the user can accept a recommended bolus delivery pattern and have a bolus dose delivered accordingly. In some embodiments, the selected bolus delivery pattern may be delivered without acceptance by the user. For example, the user can be notified prior to bolus dose delivery and can suspend the delivery or select an alternative bolus delivery pattern.

The method embodiments of providing a tailored bolus delivery pattern can be implemented in an insulin infusion device having a dispensing patch unit and a remote control unit. In some embodiments, the remote control unit may have a glucose sensing apparatus (e.g., a glucometer). The method embodiments of providing a tailored bolus delivery pattern can also be implemented in the remote control unit of the insulin infusion device, a reusable part of the dispensing patch unit of the device or both. In some embodiments, the dispensing patch unit of these method embodiments may continuously or periodically monitor BG levels and can concomitantly deliver insulin into the body. The dispensing patch unit may also include a reusable part and a disposable part. In some embodiments, the insulin dispensing and glucose sensing capabilities can be combined into a semi-closed loop system and a processor-controller apparatus may regulate the delivery of insulin bolus doses according to the sensed glucose concentration. In preferred embodiments, the bolus doses may be delivered in accordance with a tailored bolus delivery pattern.

In some embodiments, the recommended tailored bolus delivery pattern can be chosen from a plurality of pre-established tailored bolus delivery patterns based on the nutritional composition (e.g., the percentage of fat, protein and/or carbohydrates) of the food consumed (or to be consumed) by the user. In some embodiments, the dispensing patch unit may deliver a bolus dose in accordance with a tailored bolus delivery pattern selected from a plurality of pre-established bolus delivery patterns and also receive glucose levels readings. In some embodiments, the dispensing patch unit may continuously and/or periodically monitor glucose levels and deliver a bolus dose in accordance with a delivery pattern selected from a plurality of pre-established bolus delivery patterns.

Some method embodiments may provide a method for therapeutic fluid delivery. In some embodiments, the method may include determining a bolus delivery pattern based on a specified type of dietary intake to be consumed by a user (which may also be referred to as “the patient”) and delivering a therapeutic fluid into the user's body based, at least in part, on the determined bolus delivery pattern. Embodiments of the method may include determining the bolus delivery pattern by selecting the bolus delivery pattern from one or more bolus delivery patterns based on the specified type of dietary intake, where at least some of the one or more bolus delivery patterns may be determined based, at least in part, on dietary intake similar to the specific dietary intake of the user and corresponding blood glucose responses of one or more non-diabetic individuals that have consumed dietary intake similar to the specific dietary intakes of the user. The method may further include determining bolus delivery patterns based, at least in part, on users' BG responses after consuming dietary intake similar to the specific dietary intake of the user.

In some embodiments, determining a bolus delivery pattern may include computing a difference between the AUC of a graphical representation of a user's response to a dietary intake and the AUC of a graphical representation of the healthy response for the same intake.

The one or more bolus delivery patterns may include multiple bolus delivery patterns that each correspond to an associated meal type. The multiple bolus delivery patterns may include, for example, 2-10 patterns that each correspond to a different meal type. In some embodiments disclosed herein, the plurality of bolus delivery patterns may be stored in an electronic storage device.

In some embodiments, the methods disclosed herein may further include adjusting the determined bolus delivery pattern based on pharmacodynamics characteristics of the therapeutic fluid. Adjusting the determined bolus delivery pattern based on the pharmacodynamics characteristics of the therapeutic fluid may include time-shifting the determined bolus delivery pattern. In some embodiments, the method may include receiving information from the patient regarding the type of dietary intake. Some method embodiments may also include pumping insulin stored in a reservoir in a insulin infusion device into the user, graphically representing the bolus delivery pattern and/or measuring BG level of the patient.

In some embodiments, delivering the therapeutic fluid into a user based, at least in part, on the determined bolus delivery pattern may further include delivering the therapeutic fluid into a user based, at least in part, on the determined bolus delivery pattern and on the measured blood glucose level. Some methods may include measuring the blood glucose level using a glucometer or continuous glucose monitor.

Some device embodiments provide a therapeutic fluid dispensing device that delivers therapeutic fluid into a body of a user is disclosed. The device may include a controller that determines a bolus dose delivery pattern based on a specified type of dietary intake to be consumed by the user and an infusion pump to deliver the therapeutic fluid into a body of the user based, at least in part, on the determined bolus delivery pattern. Furthermore, embodiments of the device may include any of the features described in relation to the disclosed methods, as well as a user-input interface to receive at least information regarding the type of dietary intake, a reservoir in fluid communication with the infusion pump and configured to contain the therapeutic fluid, a display to represent graphically the determined bolus delivery pattern and a blood glucose sensor to measure the BG level of the patient. In some embodiments, the infusion pump may be configured to deliver therapeutic fluid into a user based, at least in part, on a determined bolus delivery pattern and a measured BG level. In some embodiments, the blood glucose sensor may include a glucometer that may be disposed in a remote control unit and further include the remote control unit. In some embodiments, the blood glucose sensor may include a continuous glucose monitor that may be disposed in a dispensing patch unit that may further include an infusion pump.

Some embodiments may include a method for tailoring a bolus delivery pattern to a patient that includes retrieving data related to a healthy response. The data may comprise one or more records, wherein each record includes a meal type and a corresponding first set of analyte concentration levels for a first period of time occurring after a meal has been consumed by a healthy individual. Such method embodiments may also include (i) initiating a bolus dose administration to the body of the patient, (ii) receiving a second set of analyte concentration levels corresponding to a second period of time, the second period of time occurring after the meal has been consumed by the patient, (iii) correlating the first set of analyte concentration levels and second set of analyte concentration levels and/or (iv) determining a bolus delivery pattern associated with the meal consumed by the patient, based on the correlation between the first set of analyte concentration levels and the second set of analyte concentration levels. Some embodiments may further comprise storing the determined bolus pattern in a memory. In some embodiments, at least one of the first set of analyte concentration levels or the second set of analyte concentration levels may be received from a continuous glucose monitor (CGM). Moreover, such method aspects may comprise visually displaying at least one of the first set of analyte concentration levels or the second set of analyte concentration levels.

In some methods embodiments, visually displaying at least one of the first set of analyte concentration levels or the second set of analyte concentration levels may comprise displaying a background image corresponding to the first set of analyte concentration levels and displaying a primary image corresponding to the second set of analyte concentration levels, wherein the primary image may be superimposed on the background image. In other embodiments, methods aspects may include visually displaying the determined bolus delivery pattern and/or administering therapeutic fluid to the body of the patient according to the determined bolus delivery pattern. Some embodiments may further comprise repeating the initiating, receiving, correlating and determining for different meal types. Some method aspects may incorporate a therapeutic fluid delivery device having (i) a reservoir retaining the therapeutic fluid, (ii) a driving mechanism dispensing the therapeutic fluid from the reservoir to the body of the patient, (iii) a controller controlling the dispensing of the therapeutic fluid, the controller being capable of correlating the first set of analyte concentration levels and the second set of analyte concentration levels, (iv) a memory storing at least one of the data related to a healthy response, the first set of analyte concentration levels, the second set of analyte concentration levels or the determined bolus delivery pattern and/or (v) a screen displaying at least one of the first set of analyte concentration levels, the second set of analyte concentration levels or the determined bolus delivery pattern. Some methods may include receiving a meal type.

In some embodiments, the disclosed methods may include dispensing a therapeutic fluid to the body of a patient according to a meal type. Such embodiments may include receiving a meal type and a content of a dietary intake to be consumed by the patient. In some embodiments, the methods may also include retrieving from a memory a pre-determined bolus pattern delivery corresponding to the meal type, determining a bolus amount of the therapeutic fluid corresponding to the content of the intake and/or dispensing the bolus amount of the therapeutic fluid to a body of a patient according to the retrieved bolus delivery pattern. Some method embodiments may include visually presenting the retrieved bolus delivery pattern. Furthermore, the meal type may be associated with a food database. In some embodiments, the content of the dietary intake may be one or more of a carbohydrate load, a GI, a fat content or a fiber content. In some embodiments, the bolus delivery pattern may correspond to one or more parameters selected from a group consisting of a CIR of the patient, an IS of the patient, a TBG of the patient, an RI of a patient and a physiological parameter of the patient.

Some device embodiments may include a graphic user interface of a drug delivery device for selecting a bolus delivery pattern. Some embodiments may include a first input element enabling the selection of a bolus amount to be delivered to a body of a patient, a second input element enabling the selection of a meal type, the meal type corresponding to a bolus delivery pattern stored in a memory of the drug delivery device and a third input element enabling initiation of drug delivery to the body of the patient corresponding to the bolus amount and bolus delivery pattern. In some embodiments, at least one of the first input element and second input element of the graphic user interface may have a scrolling functionality. The graphic user interface may further comprise a window associated with a food database that enables the selection of the meal type, according to some embodiments. The graphic user interface may also, in some embodiments, include at least one input element that visually presents at least one of a plurality of meal types, the selected meal type, the selected bolus amount, the selected bolus pattern and/or an analyte concentration level.

Embodiments of the graphic user interface may further include at least one output element visually representing a first set of analyte concentration levels, wherein the first set may correspond to the blood glucose response of a non-diabetic individual. Also, embodiments of the graphic user interface may further include at least one output element visually representing a second set of analyte concentration levels, wherein the second set of analyte concentration levels may correspond to the blood glucose response of the patient. In some embodiments, the graphic user interface may be configured to include at least one output element visually representing a background image corresponding to the first set of analyte concentration levels and a primary image corresponding to the second set of analyte concentration levels.

### BRIEF DESCRIPTION OF THE DRAWINGS

FIG. 1 is a table listing different types of foods and their respective GI.

FIG. 2 is a schematic diagram of an insulin infusion device comprising an insulin dispensing unit and a remote control unit that can be provided with a bolus delivery pattern tailoring functionality according to some embodiments described herein.

FIGS. 3a-c are graphs depicting exemplary bolus delivery patterns offered to users of currently available pumps.

FIG. 4 is a flow diagram of a procedure for providing a user with tailored bolus delivery patterns according to some embodiments described herein.

FIG. 5 shows graphs representing the healthy response and a particular user's response to a certain meal according to some embodiments described herein.

FIGS. 6a-d are diagrams of a user interface for selecting a bolus dose and tailored bolus delivery pattern according to some embodiments described herein.

FIGS. 7a-c are schematic diagrams of an insulin infusion device comprising an insulin dispensing unit, a remote control unit having a tailored bolus delivery pattern feature and a sensor according to some embodiments described herein.

FIGS. 8a-b are schematic diagrams of two configurations of an insulin infusion device comprising an insulin dispensing unit, a remote control unit having a tailored bolus delivery pattern feature and a continuous subcutaneous glucose monitor.

### DETAILED DESCRIPTION

Disclosed are methods and devices for providing tailored bolus delivery patterns of drugs (e.g., insulin). In some embodiments, a method for tailoring a bolus delivery pattern of a drug for a drug delivery device is described. This method may comprise pre-establishing meal-specific tailored bolus delivery patterns, storing the plurality of bolus delivery patterns in a memory, selecting an appropriate pre-established bolus delivery pattern in subsequent meals of the same type and adjusting a drug bolus delivery rate using the selected bolus dose delivery pattern. While the illustrative embodiments disclosed herein are described primarily in terms of determining glucose concentrations in the blood, the inventive methods and devices may be implemented to determine the concentration of other bodily analytes in other bodily tissues (e.g., ISF, or interstitial fluid) as well.

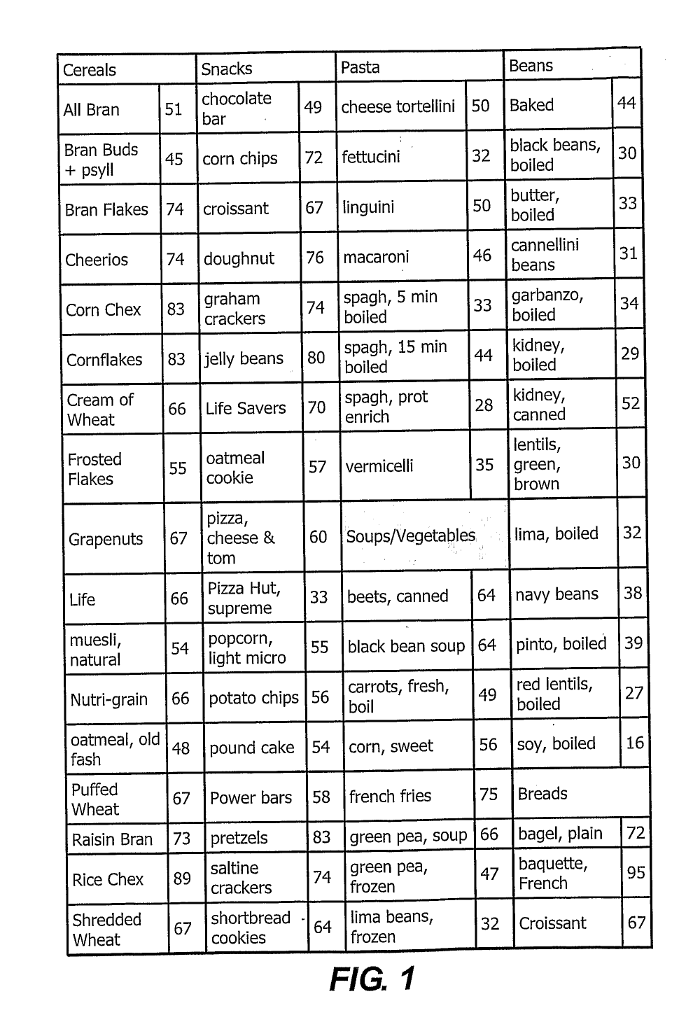

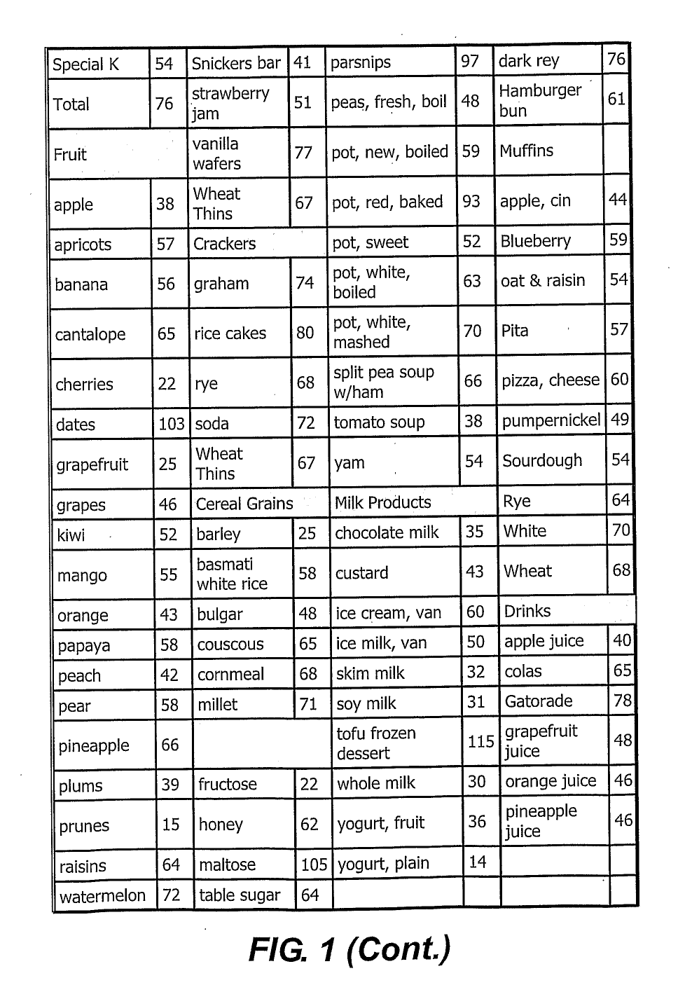

FIG. 1 is a table listing different types of foods and their respective GI's. A low GI food will release glucose more slowly and steadily. A high GI food generally causes a more rapid increase in BG levels. A meal containing a carbohydrate load having high GI would thus require insulin to be delivered immediately to counteract the rapidly absorbed carbohydrates. A meal containing a carbohydrate load having a low GI would require insulin to be delivered over a long period of time to counteract the slowly absorbed carbohydrates.

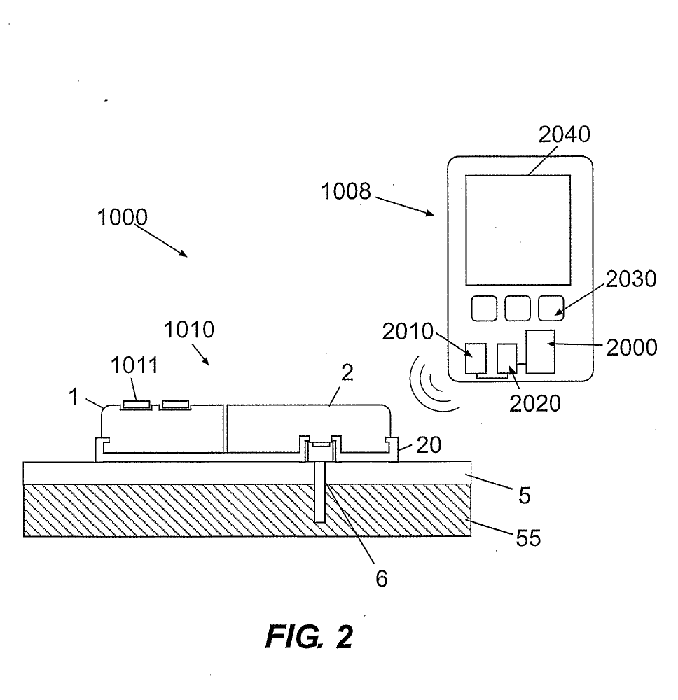

FIG. 2 shows a schematic diagram of an insulin infusion device 1000 according to some embodiments. The insulin infusion device 1000 may comprise a dispensing patch unit 1010. The unit 1010 may be secured to the user's skin 5 above the subcutaneous tissue 55. In some embodiments, a remote control unit 1008 may communicate with the dispensing patch unit 1010 to transmit (i) programming commands and/or instructions to and from the dispensing patch unit 1010, (ii) user input and output and/or (iii) acquired data.

In some embodiments, the dispensing patch unit 1010 may be detachable from the skin 5. The dispensing patch unit 1010 may also, according to some embodiments, receive a cannula 6 that penetrates the skin 5 to deliver therapeutic fluid (e.g., insulin) to the patient. The dispensing patch unit 1010 may be attached either directly to the skin 5 or to a cradle unit 20. In some embodiments, cradle unit 20 may be a low profile structure adherable to the skin 5 and enable connection and/or disconnection of the dispensing patch unit 1010 from the user. An exemplary embodiment of this type of an arrangement is described, for example, in co-owned, co-pending U.S publication no. 2008/0215035, the content of which is incorporated herein by reference in its entirety. Some embodiments of the dispensing patch unit 1010 may also contain inputs for controlling the unit 1010, including without limitation one or more buttons 1011, as shown in FIG. 2.

The dispensing patch unit 1010 may be contained in one or more housings. For example, in some embodiments, the dispensing patch unit 1010 may include a two-part housing having a reusable part 1 and a disposable part 2. This type of an arrangement is described in co-owned, co-pending U.S. patent application Ser. No. 11/397,115 and International Patent Application No. PCT/IL09/000388, the contents of which is are incorporated herein by reference in their entireties.

In some embodiments, the remote control unit 1008 may be configured to provide a tailored bolus delivery pattern. For example, as shown in FIG. 2, the remote control unit 1008 may include a tailored bolus delivery pattern module 2000, a processor 2010, a memory 2020, an input interface 2030, a display 2040 and other indication devices (not shown), including without limitation audible and vibrational output devices. The input interface 2030 may, for example, facilitate operation of the tailored bolus delivery pattern module 2000 and/or programming of the dispensing patch unit 1010.

In some embodiments, providing a tailored bolus delivery pattern may allow for the selection and/or recommendation of a bolus delivery pattern from a plurality of pre-established tailored bolus delivery patterns stored and/or offered by a software-implemented application installed on a processor (not shown) included with the dispensing patch unit 1010. According to some embodiments, the tailored bolus delivery pattern module 2000 may be located in the reusable part 1 of the dispensing patch unit 1010.

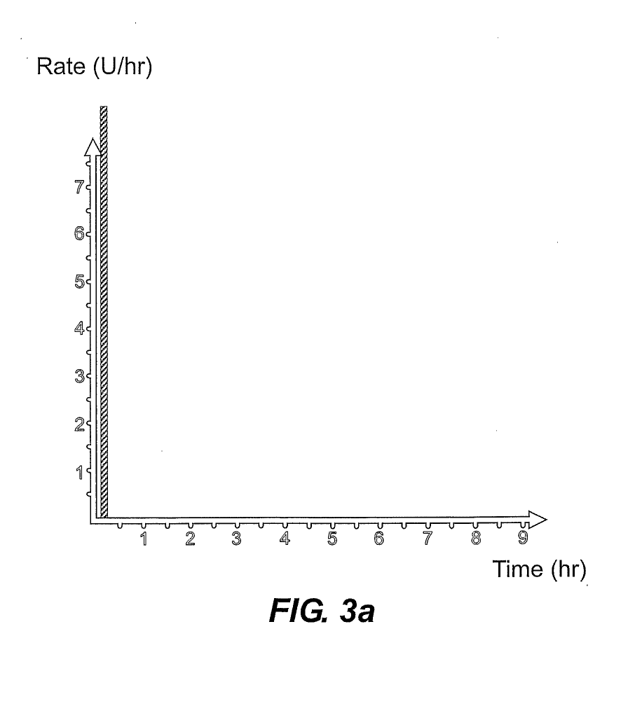

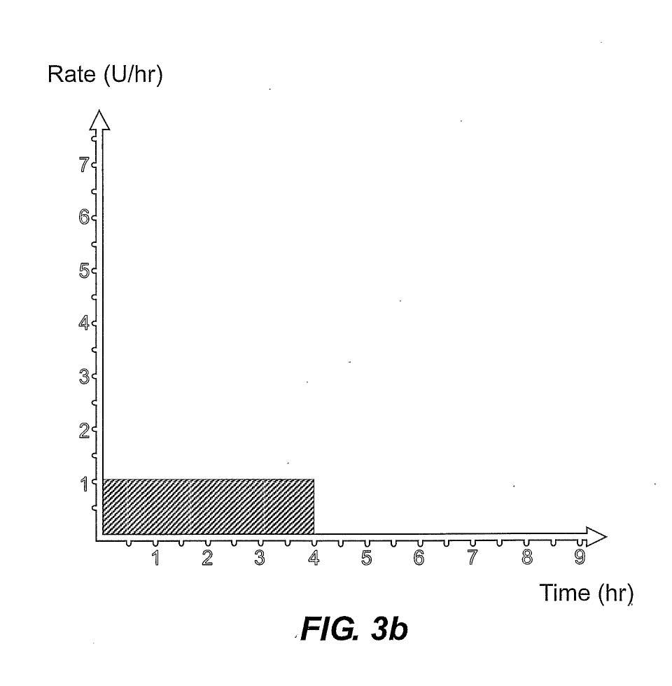

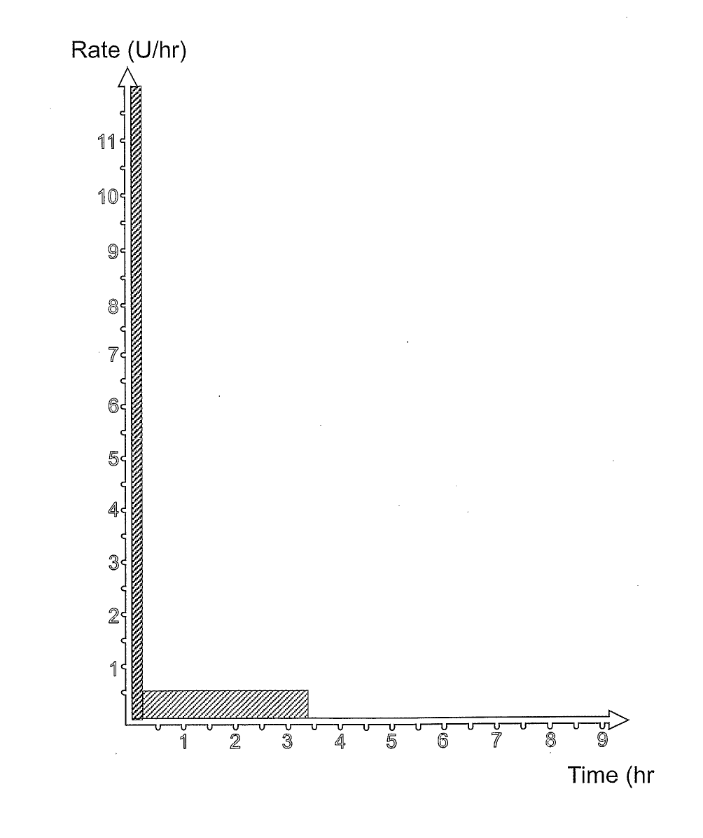

FIGS. 3a-c are graphical representations of exemplary bolus delivery patterns available for selection, recommendation and/or execution on conventional insulin pumps. The “y” axis represents the rate of bolus dose delivery (e.g., in Units/hours), and the “x” axis represents time (e.g., in hours). The area under the curve (“the AUC”) is equal to the product of the rate and the time period and constitutes the bolus amount (e.g., in Units). The AUC is substantially the same in all the patterns depicted in FIGS. 3a-c.

FIG. 3a depicts a bolus delivery pattern in which the entire bolus dose is delivered as rapidly as possible (i.e., immediate). FIG. 3b depicts a bolus delivery pattern in which the bolus dose is delivered evenly over a period of time (i.e., “extended”). In FIG. 3b, the bolus dose is delivered evenly over 4 hours. FIG. 3c depicts a bolus delivery pattern in which a portion of the bolus dose may be delivered immediately and the remaining dose delivered evenly over an extended period of time. This bolus delivery pattern may be called a combined or “dual wave” bolus dose. The user can generally set the duration of the extended portion of the bolus dose and its relative proportion. In the depicted pattern, and by way of example only, the immediate portion may comprise 60% of the total bolus dose, and the remaining 40% delivered over a period of time (e.g., 2-3 hours).

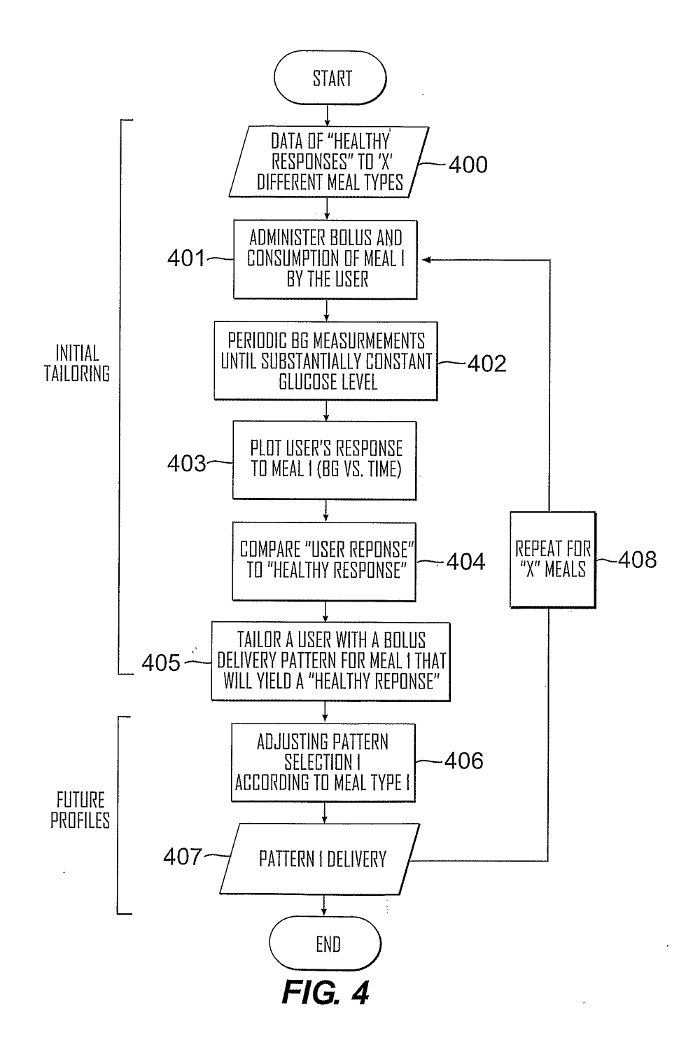

FIG. 4 shows a flow diagram of a procedure for providing tailored bolus delivery patterns according to some embodiments. Data of the healthy response to ‘x’ different meal types is provided at 400. In some embodiments, the notation ‘x’ may represent any number of different meal types (e.g., based on different GI's—high GI, medium GI, low GI and very low GI). A meal-specific tailoring procedure is performed at 401-405 according to some embodiments. For example, at 401 a user may consume a meal number 1 (e.g., one of the ‘x’ meals) and a bolus dose (e.g., a bolus dose delivered over a 60-minute time period) may be delivered to counteract the carbohydrate load of meal number 1. The bolus dose to be delivered may be computed using equations involving carbohydrate content and parameters including without limitation carbohydrate-to-insulin ratio (“CIR”) and TBG, or target blood glucose, levels. The performance of such computations is described, for example, in U.S. Pat. No. 6,936,029 to Mann et al., the content of which is incorporated herein by reference in its entirety. In some embodiments, the bolus dose level may be determined according to the selection procedure described, for example, in U.S. publication no. 2008/0234663, the content of which is incorporated herein by reference in its entirety.

Periodic (e.g., every 15 minutes) BG measurements may be performed at 402 (e.g., by a glucometer or continuous glucose monitoring (“CGM”)) after consumption of the meal number 1 until a substantially constant BG level is reached (e.g., ±15 mg/dL). In some embodiments, the user's response to the meal number 1 may be plotted at 403 as a BG versus time curve. The user's response to the meal number 1 may be compared at 404 to the healthy response for a meal of the same type as the meal number 1. As will be described in greater details below, a bolus delivery pattern is then tailored at 405 to the user so that the user's response will substantially match, or nearly match, the healthy response.

In subsequent meals of the same type as meal number 1, the appropriate pre-established tailored bolus delivery pattern will be selected at 406, and delivered at 407. This procedure can be performed, as shown at 408, for any ‘x’ number of meals.

In some embodiments, the most suitable pre-established tailored bolus delivery pattern may be based on one or more stored tailored bolus delivery patterns based on a user's input of a meal. For example, a hamburger meal with French fries and soda would correspond to one of the stored tailored bolus delivery patterns. In some embodiments, the most suitable bolus delivery pattern may be recommended based on the user's input of meal type (e.g., a meal having a high, low or intermediate GI).

In some embodiments, the bolus dose may be delivered automatically according to a selected pre-established bolus delivery pattern corresponding to a specific meal. For example, when a user selects a meal from the meals list (e.g., a “pizza meal”) the bolus dose may automatically be administered according to a corresponding tailored bolus delivery pattern. In some embodiments, the user can be recommended a particular bolus delivery pattern and may then accept or reject that recommendation. In some embodiments, the meal list can be presented alpha-numerically. The meal list may also be presented graphically on a display (e.g., display 2040 in FIG. 2) of the remote control unit 1008 or on a display (not shown) on the reusable part 1. According to some embodiments, the user may determine a bolus delivery pattern number (i.e., determine an ‘x’ number) tailored for selection of the bolus delivery pattern most appropriate for a specific meal.

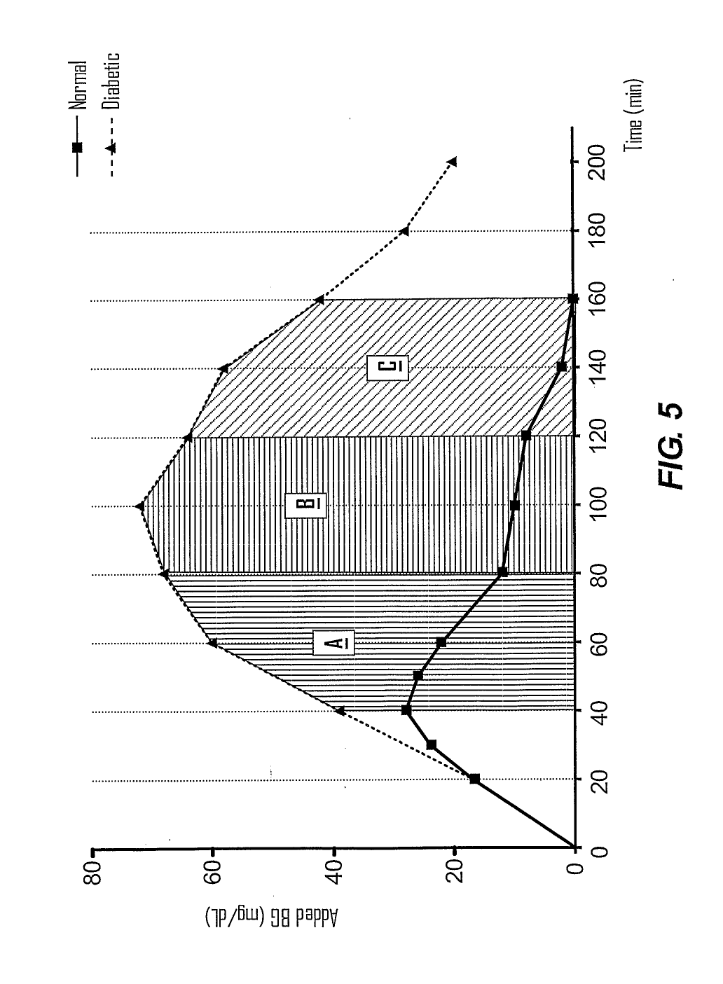

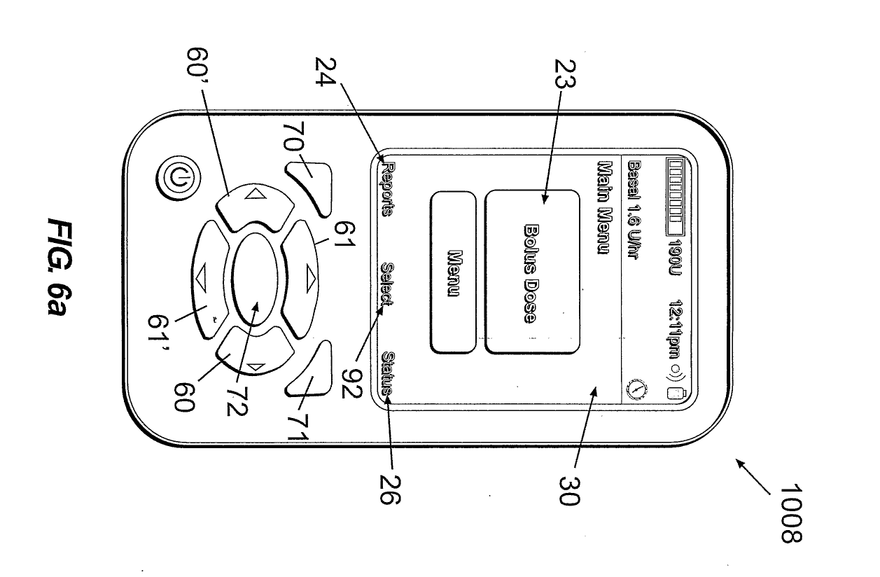

FIG. 5 shows graphs of a healthy response (“Normal” in FIG. 5) and the user's response (“Diabetic” in FIG. 5) to a certain meal type according to some embodiments. The “y” axis of the graph represents the added BG (mg/dL) (i.e., the glucose concentration added to the baseline fasting concentration due to meal consumption) and the “x” axis represents the time that has elapsed since the meal was consumed (e.g., in minutes).

In some embodiments, the procedure to tailor a bolus delivery pattern and/or provide the user with such a tailored bolus dose and pattern, based on a planned meal and individual parameters (e.g., insulin sensitivity) may include:

1. Determining data representing the healthy response and the user's response to a certain meal type and graphically representing this data, as shown in FIG. 5.

2. Dividing the time of the response pattern into n segments (n being any positive number).

3. Computing the AUC (e.g., in min*mg/dL) of an average healthy response (“AUCₕ”) in each segment.

4. Computing the AUC of the user's response (“AUCᵤ”) in each segment.

5. Determining the ideal bolus dose distribution over time (i.e., delivery rate) from the difference between the AUC of the user and the non-diabetic “AUCₕ” (i.e., AUCᵤ-AUCₕ) for each segment, namely, the bolus dose per segment required to substantially match the user's BG to that of an average BG of a non-diabetic individual.

In FIG. 5 the curve is divided, for simplicity, into three segments (segments A, B, C) according to some embodiments. Segment A starts at t=40 min. due to the relative similarity in the curves of the user's response and the healthy response immediately after meal consumption. In some embodiments, a positive correction bolus dose (i.e., a dose in addition to what has been delivered as an extended bolus) or a negative correction bolus dose (i.e., less than what has been delivered) can be computed per segment. For segment A, (AUCᵤ₄₀₋₈₀-AUCₕ₄₀₋₈₀)/IS=Insulin *t_{A}, [UI*min] where AUCₕ₄₀₋₈₀ may represent the AUC of the average healthy response between 40 and 80 minutes after the meal, AUCᵤ₄₀₋₈₀ is the area under the curve of the user's response between 40 and 80 minutes after the meal, t_{A} is the duration of time of segment A and IS is the insulin sensitivity of the user (i.e., the BG level lowered by one unit of insulin). The correction insulin dose for segment A therefore equals (AUCᵤ₄₀₋₈₀-AUCₕ₄₀₋₈₀)/(IS*t_{A}). For segment B, (AUCᵤ₈₀₋₁₂₀-AUCₕ₈₀₋₁₂₀)/IS=Insulin*t_{B}, where t_{B} is the duration of time of segment B. The correction insulin dose for segment B therefore equals (AUCᵤ₈₀₋₁₂₀-AUCₕ₈₀₋₁₂₀)/(IS*t_{B}). For segment C, (AUCᵤ₁₂₀₋₁₆₀-AUCₕ₁₂₀₋₁₆₀)/IS=Insulin *t_{C}, where t_{C} is the duration of time of segment C. The correction insulin dose for segment C therefore equals (AUCᵤ₁₂₀₋₁₆₀-AUCₕ₁₂₀₋₁₆₀)/(IS*t_{C}).

According to some embodiments, the total bolus dose may be redistributed over the delivery time period according to the computed correction bolus doses for each segment. In some embodiments, the absolute bolus doses per segment may then be expressed as a percentage of the total bolus dose per segment, thus providing a bolus delivery pattern tailored to the user for the corresponding meal. Subsequent meals of similar type (e.g., similar dietary or nutritional content), even of different sizes, may be counteracted by bolus doses delivered according to the tailored bolus delivery pattern.

In some embodiments, bolus dose administration time may be adjusted and/or corrected in accordance with the pharmacodynamics of a specific insulin type . For example, if insulin Lispro is used, which has an onset time of approximately 15 minutes from administration, distribution of the bolus dose may be time shifted by 15 minutes to match the action of the insulin lispro. In some embodiments, the time shift may consider additional pharmacodynamics characteristics including without limitation the time until peak activity is obtained, the total duration of action of the delivered bolus dose.

Using the abovementioned procedure, a bolus delivery pattern may be tailored to the user based on meal type. The more segments into which a pattern is divided and the more BG measurements performed, the more accurate the tailored pattern will become. With that said, however, the utility of additional segments in accurately tailoring a bolus delivery pattern will depend on the state of the user and the specific meal type. For example, not all meals may precisely match a pre-established bolus delivery pattern.

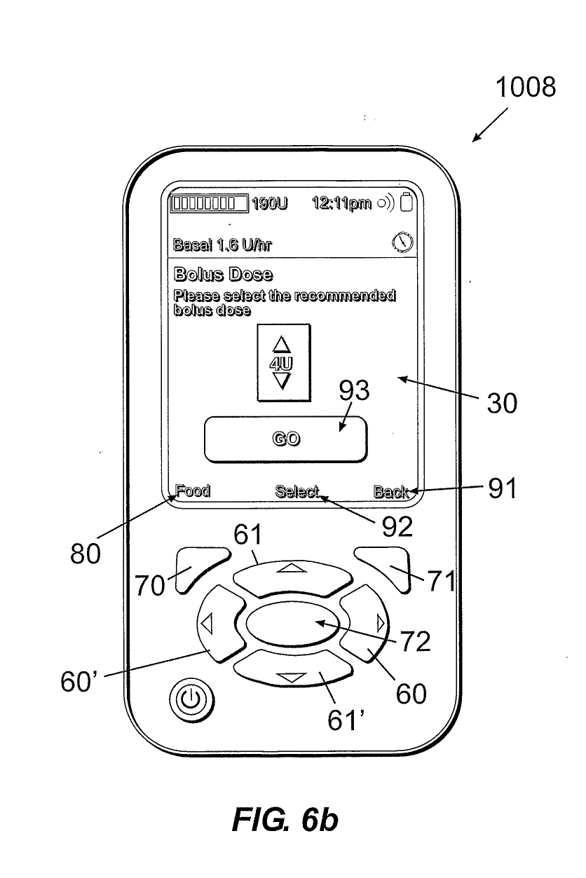

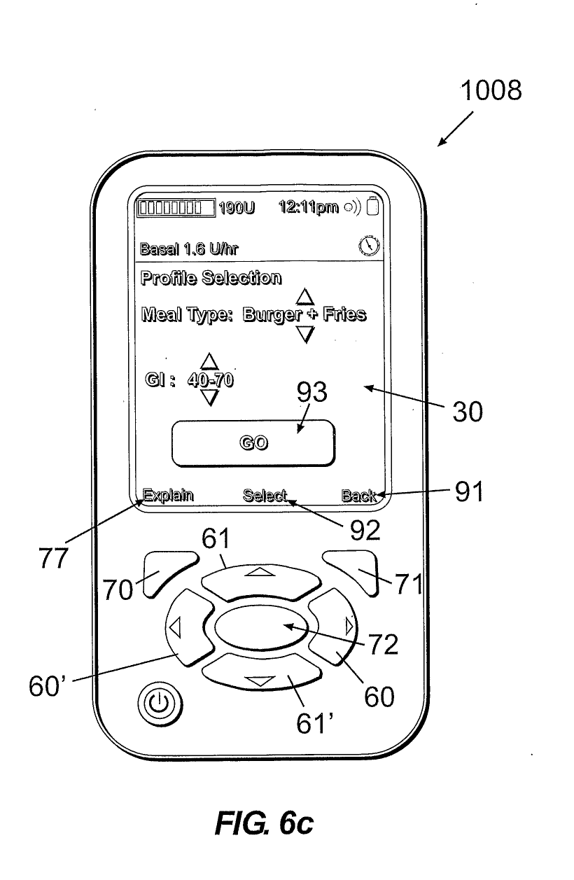

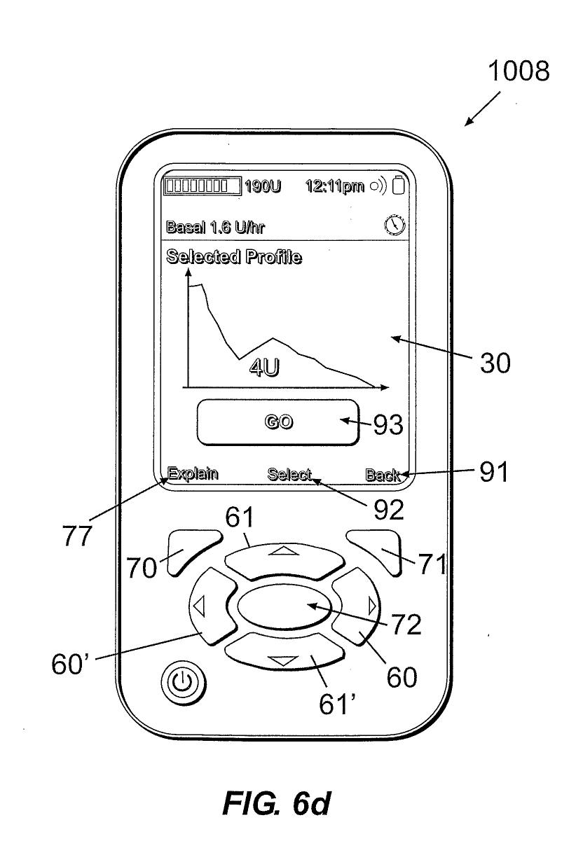

FIGS. 6a-d show user interfaces for selecting bolus doses and tailored bolus delivery patterns. In some embodiments, the user interface may be displayed on a screen 30 of the remote control unit 1008, a screen on the dispensing patch unit 1010 (not shown) or screens on both remote control unit 1008 and dispensing patch unit 1010. The user interface can be provided with a plurality of navigation windows for data input according to some embodiments. For example, FIG. 6a shows an example of a main window of the user interface. If the user selects “Bolus Dose” 23, then the window shown in FIG. 6b can be displayed. In some embodiments, auxiliary windows may be provided and/or selected by pressing, for example, soft keys 70 and/or 71. Some embodiments may have a window for downloading last bolus dose data displayed by selecting the “Reports” function 24 (shown in FIG. 6a). In response to selecting the “Status” function 26, the user may display data regarding ongoing bolus doses. The user may also display data regarding analyte (e.g., glucose) concentration levels by selecting the corresponding function.

FIG. 6b also shows an example of a window which can be used for selection of a bolus dose. According to some embodiments, the bolus dose can be recommended by the insulin infusion device 1000 itself based on personal parameters (e.g., CIR, IS, CBG or TBG) associated with a specific user. Types of input data used for determining the bolus dose are described, for example, in co-owned, co-pending U.S. publication no. 2008/0234663, the content of which is incorporated herein by reference in its entirety. According to some embodiments, the most frequently used and/or an averaged bolus dose level, selected during a specific time interval, can be presented at this window and presented as a first or “default” choice. This feature may be especially beneficial for users with routine daily intakes. In some embodiments, the “Food” function 80 may also be provided. Selecting this function using, for example, the soft key 70, can provide the user with a food database displaying different types of foods and their GI and/or carbohydrate load and/or fat content.

FIG. 6c shows an example of a window in which the user selects the relevant meal type for subsequent adjustments of the optimal bolus delivery pattern. In certain embodiments, the meal type can be described in terms of meal type or GI range. In some embodiments, the “Explain” function 77 can also be provided. Selecting this function using, for example, soft key 70, can provide the user with a short, general explanation of the meal content and its relevance to one of the pre-established tailored bolus delivery pattern.

In a subsequent window, depicted in FIG. 6d, the pre-established tailored bolus delivery pattern associated with the meal type selected in the window depicted in FIG. 6c and the dose selected in FIG. 6b, may be graphically displayed according to some embodiments. The AUC of the displayed bolus delivery pattern represents the bolus dose that needs to be delivered. This user interface also illustrates the “GO,” “Explain” and “Back” functions. In some embodiments, selecting the “GO” function may initiate delivery of a bolus dose (e.g., the insulin bolus) according to data represented graphically in the user interface display.

According to some embodiments, the user may be prompted to select a meal-type dependent pattern via the window displays depicted in FIGS. 6c and 6d and to initiate bolus dose delivery using the “GO” function shown in FIG. 6d. In some embodiments, applying the tailored bolus delivery pattern feature is not required to initiate a bolus dose delivery. That is, in some embodiments, a bolus dose does not have to be delivered according to a bolus delivery pattern, tailored or otherwise.

In some embodiments, where the user interface is provided in the remote control unit 1008, the user can navigate between displayed icons using horizontal 60, 60′ and vertical 61, 61′ buttons. Selection of a particular bolus dose and/or a bolus delivery pattern may be made by scrolling up and down through a list of possible doses and patterns to choose from, according to some embodiments. The user may also press the soft keys, namely, left key 70, right key 71 and center key 72 of the remote control unit 1008. These soft keys, when pressed, function to select certain functions on the display. In the given example of FIG. 6c, upon pressing the left soft key 70, an “Explain” function 77 is activated. Upon pressing the right soft key 71, the “Back” function 91, which transfers the display to the previous entered window, is performed. Upon pressing the central soft key 70, the “GO” function 93 is performed and transfers the user to the next window.

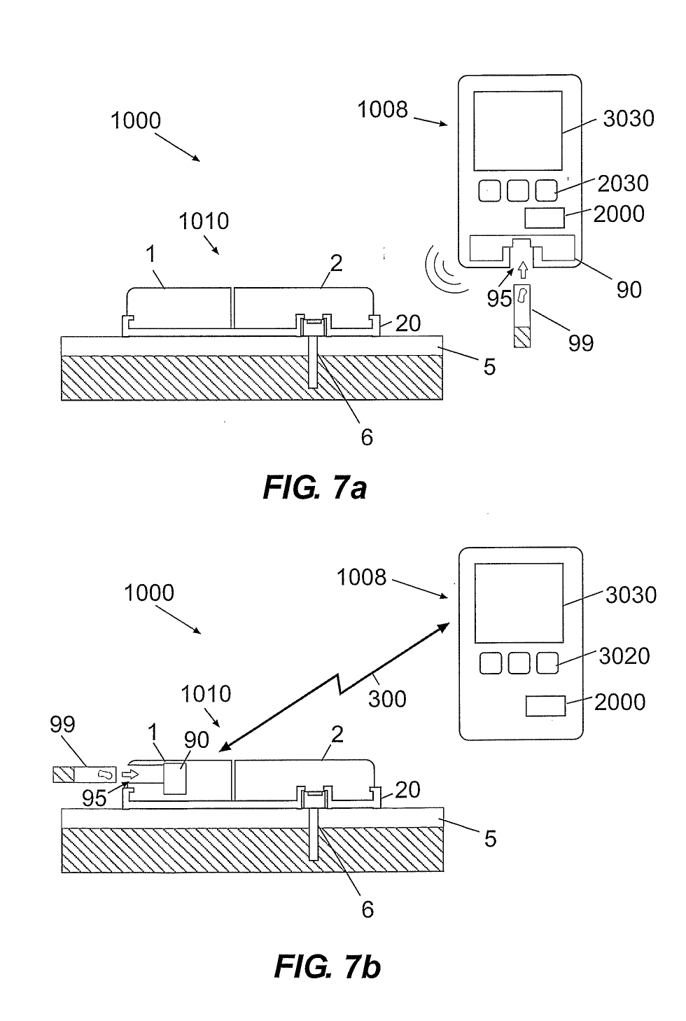

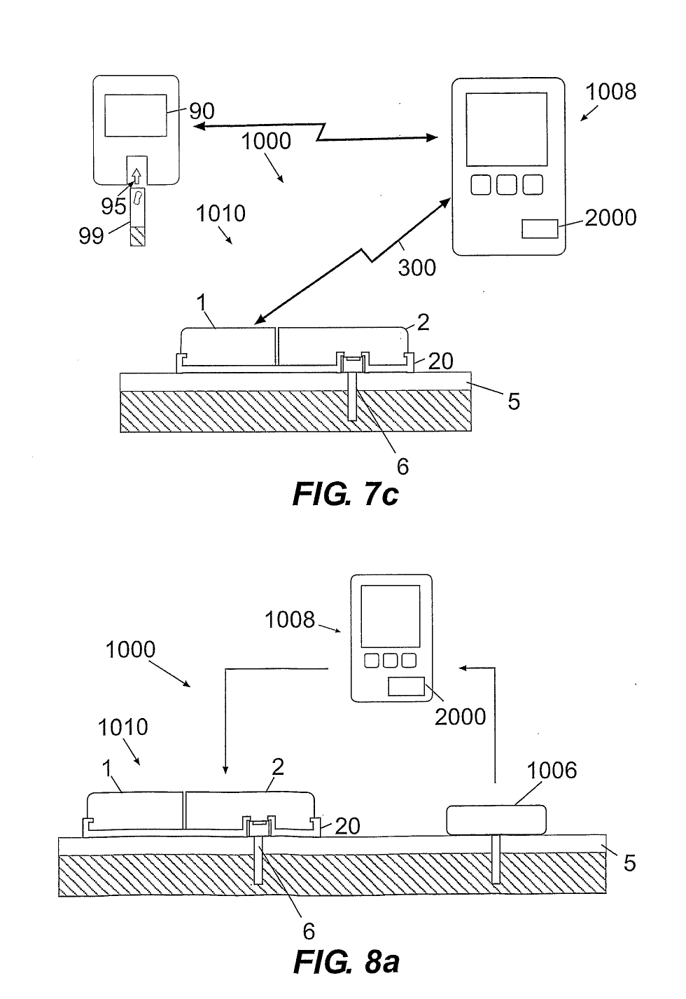

FIGS. 7a-c show schematic diagrams of three different embodiments of an insulin infusion device 1000. The insulin infusion device 1000 may include a dispensing patch unit 1010, a remote control unit 1008 implemented with a functionality to provide a tailored bolus delivery pattern and a sensor 90 (e.g., “glucometer”) to perform BG monitoring for providing a tailored bolus delivery pattern. FIG. 7a shows a sensor 90 located in the remote control unit 1008. In some embodiments, the sensor 90 may comprise an opening 95 for receiving of a test strip 99. The user may extract blood from the body, place a blood drop on the test strip 99 and insert the strip 99 into the opening 95, according to some embodiments. The glucose readings may be shown on a display 3030 of the remote control unit 1008. FIG. 7b shows a sensor 90 located in the reusable part 1 of the dispensing patch unit 1010, according to some embodiments. FIG. 7c shows an embodiment in which glucose readings are directly or remotely received from an independent sensor 90.

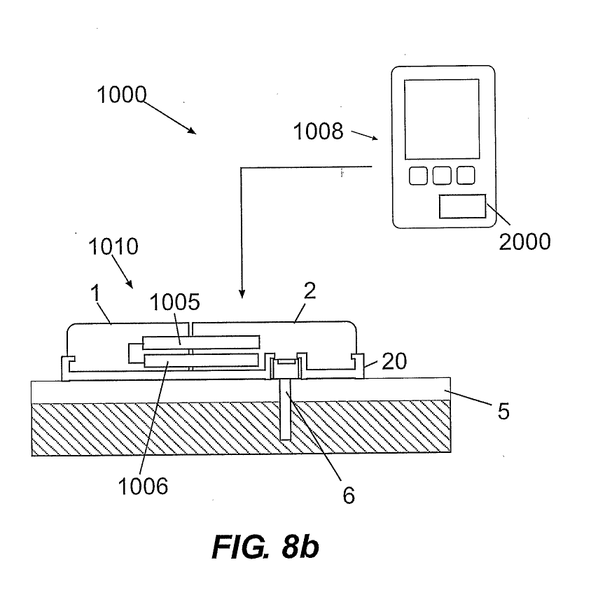

FIGS. 8a-b show schematic diagrams of two embodiments of an insulin infusion device 1000. The insulin infusion device 1000 may include a dispensing patch unit 1000, a remote control unit 1008 configured to include a tailored bolus delivery pattern module 2000 and a continuous subcutaneous glucose monitor 1006 required for the glucose measurements needed for performing the tailored bolus delivery pattern procedure. Specifically, FIG. 8a shows an embodiment in which the current BG concentration can be received from an independent continuous subcutaneous glucose monitor 1006. FIG. 8b shows an embodiment in which the continuous subcutaneous glucose monitor 1006 is located in the dispensing patch unit 1010 of the insulin infusion device 1000. As disclosed in co-owned, co-pending PCT Application Nos. PCT/IL07/000163, PCT/IL07/001579, and PCT/IL08/001521, the contents of which are incorporated herein by reference in their entireties, the insulin dispensing apparatus 1005 and continuous subcutaneous glucose monitor 1006 shown in FIG. 8b may constitute, in the illustrated embodiment, a common insulin delivery device and share a single cannula 6 for both dispensing and sensing according to some embodiments. In some embodiments, the continuous subcutaneous glucose monitor 1006 and the insulin dispensing apparatus 1005 may have separate cannulae that penetrate the skin 5 and reside in the subcutaneous tissue.

In some embodiments, the device 1000 can be configured to operate as a semi-closed loop system. In a semi-closed system, the feedback and control between sensor measurements and associated therapeutic fluid release can be partially automatic. For example, the release of therapeutic fluid in a basal rate can be automatically controlled by a processor based on analytes measurements, while the release of bolus doses of therapeutic fluid may be delivered according to a user's input. In some embodiments, insulin can automatically be dispensed according to continuous monitoring of glucose levels and according to additional pre-meal bolus user inputs (semi-closed loop). The tailored bolus delivery pattern module 2000 shown in FIG. 8b can be used for bolus inputs in the semi-closed loop system.

Various embodiments of the subject matter described herein may be realized in digital electronic circuitry, integrated circuitry, specially designed ASICs (application specific integrated circuits), computer hardware, firmware, software, and/or combinations thereof. These various embodiments may include being implemented in one or more computer programs that are executable and/or interpretable on a programmable system including without limitation (i) at least one programmable processor, which may be special or general purpose, coupled to receive data and instructions from, and to transmit data and instructions to, a storage system, (ii) at least one input device and/or (iii) at least one output device.

These computer programs (also known as programs, software, software applications or code) may include machine instructions for a programmable processor and may be implemented in a high-level procedural and/or object-oriented programming language and/or in assembly/machine language. As used herein, the term “machine-readable medium” refers to any computer program product, apparatus and/or device (e.g., magnetic discs, optical disks, memory or Programmable Logic Devices (PLDs)) used to provide machine instructions and/or data to a programmable processor, including without limitation a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user, a keyboard and a pointing device (e.g., a mouse or a trackball) for providing input to the computer. Other types of devices may also be used to enable interaction with a user. For example, output to the user may be provided in any form, including without limitation sensory feedback (e.g., visual feedback, auditory feedback or tactile feedback) and input from the user may be received in any form, including without limitation acoustic, speech or tactile input.

The subject matter described herein may be implemented in a computing system that includes a back-end component (e.g., as a data server), a middleware component (e.g., an application server) a front-end component (e.g., a client computer having a graphic interface or a Web browser through which a user may interact with the subject matter described herein), or any combination of back-end, middleware or front-end components. The components of the system may be interconnected by any form of digital data communication (e.g., a communication network). Examples of communication networks include a local area network (“LAN”), a wide area network (“WAN”) and the Internet.

The computing system may also include clients and servers. A client and server are generally remote from each other and typically interact through a communication network. The relationship of client and server may arise by virtue of computer programs running on the respective computers and having a client-server relationship.

Any and all references to publications or other documents, including but not limited to, patents, patent applications, articles, webpages, books, etc., presented in the present application, are herein incorporated by reference in their entirety.

Example embodiments of the methods and devices of the present disclosure have been described herein. As noted elsewhere, these embodiments have been described for illustrative purposes only and are not limiting. Other embodiments are possible and are covered by the present disclosure. For example, the logic flow depicted in the accompanying figures and described herein do not require the particular order shown, or sequential order, to achieve desirable results. Such embodiments will be apparent to persons of ordinary skill in the relevant art(s) based on the teachings contained herein. Thus, the breadth and scope of the disclosure should not be limited by any of the above-described embodiments but should be defined only in accordance with the following claims and their equivalents.

1.-55. (canceled)

56. A therapeutic fluid dispensing device to deliver therapeutic fluid into the body of a patient, the device comprising:
a controller having instructions operable thereon configured to determine a bolus delivery pattern based on a specific dietary intake of the patient; and
an infusion pump to deliver the therapeutic fluid into the body of the patient based, at least in part, on the determined bolus delivery pattern,
wherein the instructions are further configured to select the bolus delivery pattern from a plurality of bolus delivery patterns,
at least one of the bolus delivery patterns being determined based on at least one of a dietary intake similar to the specific dietary intake of the patient,
and other delivery patterns corresponding to blood glucose responses of one or more non-diabetic individuals that have consumed dietary intakes similar to the specific dietary intake of the patient.

57. The device of claim 56, wherein the controller is configured to determine the bolus delivery patterns based additionally on blood glucose responses of the patient after the patient has consumed dietary intakes similar to the specific dietary intakes.

58. The device of claim 57, wherein for each of the bolus delivery patterns, the controller is configured to compute a difference between an area under the curve of a graphical representation of the response of the patient to a respective type of dietary intake and an area under the curve of a graphical representation of the response of the one or more non-diabetic individuals to the respective type of dietary intake.

59. The device of claim 56, wherein the specific dietary intake comprises a glycemic index representative of the effect of the carbohydrate content of food corresponding to the type of dietary intake on blood glucose levels in individuals consuming the food.

60. The device of claim 56, further comprising a blood glucose sensor to measure the blood glucose level of the patient.

61. The device of claim 60, wherein the blood glucose sensor includes a glucometer.

62. The device of claim 61, wherein the glucometer is disposed in a remote control that further includes the controller.

63. The device of claim 60, wherein the blood glucose sensor includes a continuous glucose monitor.

64. The device of claim 63, wherein the continuous glucose monitor is disposed in a dispensing patch unit that includes the infusion pump.

65. A method for delivering therapeutic fluid, the method comprising:
determining a bolus delivery pattern based on a specific dietary intake of a patient; and
delivering a therapeutic fluid into the body of the patient based, at least in part, on the determined bolus delivery pattern,
wherein determining the bolus delivery pattern includes selecting a bolus delivery pattern from a plurality of bolus delivery patterns,
wherein at least one of the bolus delivery patterns being determined based on at least one of dietary intakes similar to the specific dietary intake of the patient, and other delivery patterns corresponding to blood glucose responses of one or more non-diabetic individuals that have consumed dietary intakes similar to the specific dietary intake of the patient.

66. The method of claim 65, wherein the at least one bolus delivery pattern is additionally based on one or more blood glucose responses from the patient after the patient has consumed dietary intakes similar to the specific dietary intake.

67. The method of claim 66, wherein determining the at least one bolus delivery pattern comprises computing a difference between an area under the curve of a graphical representation of the response of the patient to a respective type of dietary intake and an area under the curve of a graphical representation of the response of the one or more non-diabetic individuals to the respective type of dietary intake.

68. The method of claim 65, further comprising adjusting the determined bolus delivery pattern based on pharmacodynamics characteristics of the therapeutic fluid.

69. The method of claim 68, wherein adjusting the determined bolus delivery pattern based on the pharmacodynamics characteristics of the therapeutic fluid includes time-shifting the determined bolus delivery pattern.

70. The method of claim 65, wherein delivering the therapeutic fluid into the body of the patient comprises pumping insulin stored in a reservoir in a fluid infusion device into the body of the patient.

71. The method of claim 65, further comprising graphically representing the bolus delivery pattern.

72. A method for tailoring a bolus pattern to a patient, the method comprising:
retrieving data related to a healthy response, the data comprising one or more records, each record including a meal type and a corresponding first set of analyte concentration levels for a first period of time occurring after a meal has been consumed by a healthy individual;
initiating a bolus dose administration to the body of the patient;
receiving a second set of analyte concentration levels corresponding to a second period of time, the second period of time occurring after the meal has been consumed by the patient;
correlating the first set of analyte concentration levels and second set of analyte concentration levels; and
determining a bolus delivery pattern associated with the meal consumed by the patient, based on the correlation between the first set of analyte concentration levels and the second set of analyte concentration levels.

73. The method of claim 72, further comprising visually displaying at least one of the first set of analyte concentration levels and the second set of analyte concentration levels.

74. The method of claim 73, wherein visually displaying the at least one of the first set of analyte concentration levels and the second set of analyte concentration levels comprises:
displaying a background image corresponding to the first set of analyte concentration levels, and
displaying a primary image corresponding to the second set of analyte concentration levels,
wherein the primary image is superimposed on the background image.

75. The method of claim 72, further comprising visually displaying the determined bolus delivery pattern.

76. The method of claim 72, comprising providing a therapeutic fluid delivery device including:
a reservoir retaining the therapeutic fluid;
a driving mechanism dispensing the therapeutic fluid from the reservoir to the body of the patient;
a controller controlling the dispensing of the therapeutic fluid, the controller is further capable of correlating the first set of analyte concentration levels and the second set of analyte concentration levels;
a memory storing at least one of the data related to a healthy response, the first set of analyte concentration levels, the second set of analyte concentration levels and the determined bolus delivery pattern; and
a screen displaying at least one of the first set of analyte concentration levels, the second set of analyte concentration levels and the determined bolus delivery pattern.
